(19) United States
(12) Patent Application Publication
(10) Pub. No.: US 2018/0207398 A1
(43) Pub. Date: Jul. 26, 2018

(54) CLOSED SYSTEM FOR UMBILICAL VEIN ACCESS

(71) Applicant: pfm Medical, Inc., Carlsbad, CA (US)

(72) Inventor: Jiaye Jho, Carlsbad, CA (US)

(21) Appl. No.: 15/411,387

(22) Filed: Jan. 20, 2017

(51) Int. Cl. A61M 25/00; A61M 39/10

(52) U.S. Cl. CPC A61M 25/0097; A61M 2039/1072; A61M 2240/00; A61M 39/10

(57) ABSTRACT

Methods and devices are configured for accessing an umbilical vein of a human. The umbilical vein catheter includes a proximal access port that is closed with a septum. The septum provides a closed system to reduce the risk of air embolism and intraluminal infection via the catheter.

### BACKGROUND

Disclosed herein is a closed system for umbilical vein catheterization and infusion.

In the situation of a pre-term, premature, or new born human baby, it can be difficult to obtain reliable vascular access and it can be particularly difficult to obtain central venous access. A newborn's umbilical vein is often used to gain vascular access using an umbilical vein catheter as the umbilical vein has a relatively convenient size for insertion of such a catheter.

However, conventional umbilical vein catheters are open ended and are therefore susceptible to infection. That is, such conventional umbilical vein catheters have proximal hubs that communicate with an internal lumen of the catheter and that are open to the environment. Consequently, the opens hubs make the catheters prone to intraluminal infection, air embolism, and bleeding due to accidental disconnect with the catheter.

In view of the foregoing, there is a need for improved systems that provide access to an umbilical vein of a pre-term, premature, or new born human baby.

### SUMMARY

The disclosed system is a closed system umbilical vein catheter with a relatively low profile and relatively small footprint that facilitate insertion of the catheter into an umbilical vein. The umbilical vein catheter includes a proximal access port that is closed with a septum. The septum provides a sealed, closed system to reduce the risk of air embolism and intraluminal infection via the catheter. Unlike a regular implantable port, the port in the disclosed system remains outside the patient's body for ease of access after the catheter has been coupled to the umbilical vein. Further, the port of the system is pre-connected instead of requiring attachment during the procedure.

Other features and advantages should be apparent from the following description of various embodiments, which illustrate, by way of example, the principles of the disclosure.

### BRIEF DESCRIPTION OF THE DRAWINGS

FIG. 1 shows a perspective view of an umbilical vein access system.

FIG. 2 shows an example access port of the system.

FIG. 3 shows an exemplary anatomy of a human infant.

### DETAILED DESCRIPTION

Disclosed are methods, systems, and devices for accessing an umbilical vein of a human. The umbilical vein catheter includes a proximal access port that is closed with a septum. The septum provides a closed system to reduce the risk of air embolism and intraluminal infection via the catheter.

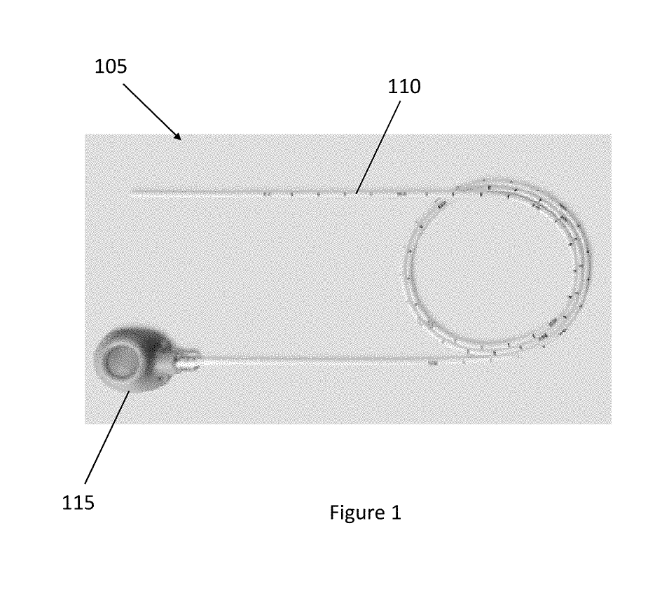

FIG. 1 shows an umbilical vein catheter system 105, which includes a catheter 110 formed of an elongated body having a proximal end and a distal end. The elongated body has an internal lumen that forms a distal opening at the distal region or distal end of the elongated body. The internal lumen also forms a proximal opening at the proximal end or proximal region of the elongated body. The internal lumen is a passageway through which fluid and/or material can flow via the distal and proximal openings. The elongated body is sized and shaped to fit within and to be at least partially inserted into or otherwise coupled to an umbilical vein of an infant, as described in more detail below. When coupled to the umbilical vein, the umbilical vein catheter system 105 provides access to the umbilical vein and the vasculature of the infant, as described in detail below.

With reference still to FIG. 1, a port 115 is positioned at the proximal end of the elongated body. The port 115 fluidly communicates with the proximal opening of the elongated body and provides a proximal access location via which a clinician can access the internal lumen of the catheter 110. In an embodiment, the port 115 is fixedly attached to the catheter 110 in that it cannot be removed from the catheter 110 without damaging or breaking the catheter 110 or affecting the functionality of the catheter. In another embodiment, the port 115 is removably attached to the catheter 110 such that the port 115 can be removed and re-attached to the catheter 110 without damaging or breaking the catheter 110.

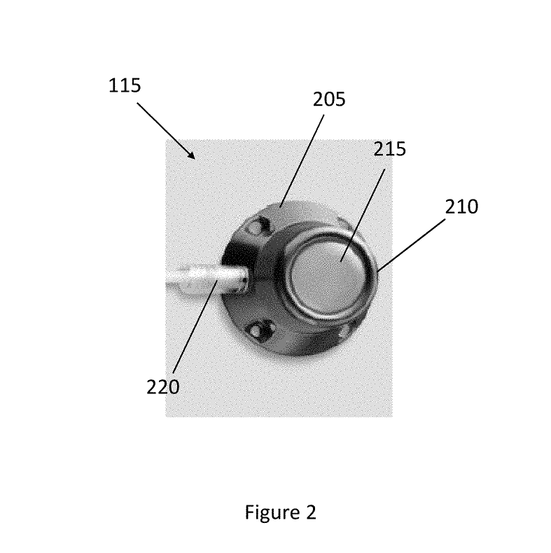

FIG. 2 shows an enlarged view of an example access port 115. The access port 115 may be formed of a cap and a base that collectively form a housing in which a septum may be positioned to form a reservoir within the housing. The access port 115 includes a base 205, a cap 210 attached to the base 205, a septum 215 positioned on or within the cap 210, and an outlet stem 220. The outlet stem 220 couples to the proximal end of the catheter 110 to provide fluid communication between the reservoir of the access port 115 and the internal lumen of the catheter. In an embodiment, the outlet stem 220 can be removably attached to the catheter 110 such that the catheter can be removed and re-attached to the access port 115 via the stem 220. In another embodiment, the outlet stem 220 is fixedly attached to the catheter 110 such that the catheter cannot be removed from the access port 115.

The cap 210 and base 205 couple to the septum 215 such that the septum 215 is captured between the cap 210 and the base 205. The reservoir is an internal cavity inside the housing formed by the cap 210 and the base 205. The cap 210 may include an opening through which a portion of the septum 215 extends and the base 205 may include a recess in which at least a portion of the septum 215 is seated. The septum is configured to be pierced by a cannula such that fluid may be injected into the reservoir via the cannula. In this manner, fluid can be injected into the blood vessel to which the catheter 110 is attached via the access port.

The dimensions of the system may vary. In a non-limiting example, the catheter 110 is less than 5 French in outer diameter and is made of polyurethane. The port may have dimensions of about 23 mm length×17.8 width mm×10 mm height. In another embodiment, the port has dimensions of about 24 mm length×20.5 width mm×10.3 mm height. It should be appreciated that these dimensions are examples and that the dimensions of the system may vary.

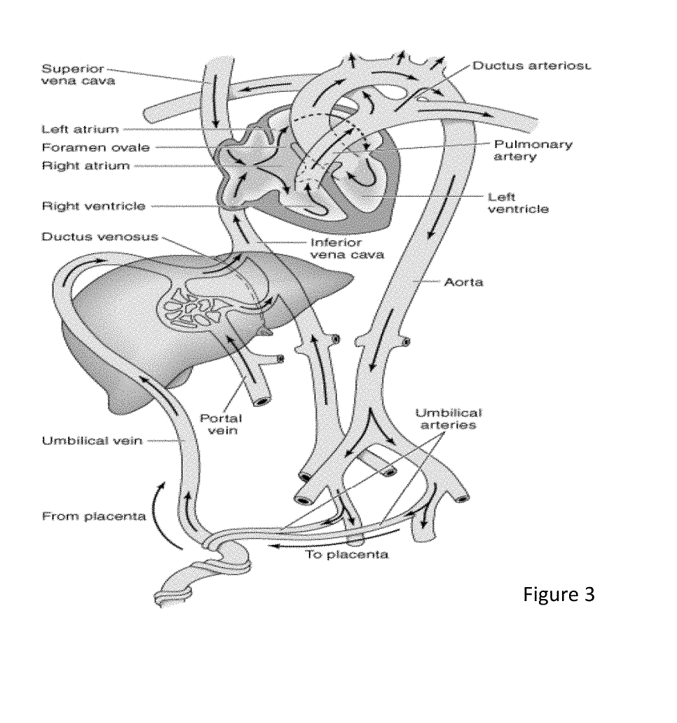

FIG. 3 shows an exemplary anatomy of an infant in the region of the umbilical vein. The umbilical vein is in the range of about 2-3 cm long and 4-5 mm in diameter although the dimensions can vary. From the umbilicus, it passes cephalad through the falciform ligament and to the right. After giving off several large intra-hepatic branches it joins the left branch of the portal vein. The ductus venosus arises from the point where the umbilical vein joins the left portal vein

There is now described a non-limiting, example process of inserting the catheter 110 into an umbilical vein of an infant. In an initial step, a clinician places a suture on the base of an umbilical cord and ties the umbilical cord to prevent excess bleeding upon cutting of the umbilical cord. The clinician then cuts the umbilical cord and inspects the anatomy to identify the blood vessels in the cut umbilical cord.

The clinician then stabilizes and exposes the vessels. Using a fine dilator, the clinician then eases the vessel open and cannulates the vessel toward the lower body. In an embodiment, the use of the catheter is optional and the clinician can insert the catheter directly into the open vein. The clinician then applies gentle, steady pressure to insert the catheter 110 into the blood vessel. The clinician can then aspirate to ensure a “flashback” arterial blood from the UAC. The umbilical venous catheter 110 is inserted into the umbilical vein such that a desired length of a distal region of the catheter is inside the umbilical vein. In this state, the catheter 110 provides access to the vein via the port 115 and the internal lumen of the catheter 110. The catheter 110 can be sutured separately and fixed in place.

The clinician can aspirate, infuse or inject material from or into the catheter using the access port 115. In this regard, the clinician can insert a cannula through the septum of the access port such that the cannula communicates with the reservoir of the access port 115. The clinician can then use the cannula to aspirate or infuse fluid from or into the catheter via the access port 115.

As mentioned, the port 115 remains outside the infant's body (and/or outside the mother's body) after the catheter 110 has been coupled to the umbilical vein. Because the port 115 is sealed, there is a reduced likelihood of infectious material being introduced into the umbilical vein via the catheter. The catheter 110 may remain coupled to the umbilical vein for a period of time, such as for about ten days. The duration for which the catheter remains coupled to the umbilical vein can be as needed and can also be dictated by the clinician.

While this specification contains many specifics, these should not be construed as limitations on the scope of an invention that is claimed or of what may be claimed, but rather as descriptions of features specific to particular embodiments. Certain features that are described in this specification in the context of separate embodiments can also be implemented in combination in a single embodiment. Conversely, various features that are described in the context of a single embodiment can also be implemented in multiple embodiments separately or in any suitable sub-combination. Moreover, although features may be described above as acting in certain combinations and even initially claimed as such, one or more features from a claimed combination can in some cases be excised from the combination, and the claimed combination may be directed to a sub-combination or a variation of a sub-combination. Similarly, while operations are depicted in the drawings in a particular order, this should not be understood as requiring that such operations be performed in the particular order shown or in sequential order, or that all illustrated operations be performed, to achieve desirable results.

Although embodiments of various methods and devices are described herein in detail with reference to certain versions, it should be appreciated that other versions, embodiments, methods of use, and combinations thereof are also possible. Therefore the spirit and scope of the appended claims should not be limited to the description of the embodiments contained herein.

1. A system for accessing an umbilical blood vessel, comprising:
an elongated catheter having a proximal end and a distal end and an internal lumen, the elongated catheter also having a distal opening that communicates with the internal lumen and a proximal opening that communicates with the internal lumen, and wherein a distal region of the catheter is sized and shaped to be inserted into the umbilical blood vessel;
an access port on the proximal end of the elongated catheter, the access port providing access to the internal lumen of the catheter, wherein the access port includes a seal that prevents entry of fluid into the catheter.

2. A system as in claim 1, wherein the access port is removably attached to the catheter.

3. A system as in claim 1, wherein the access port is fixedly attached to the catheter.

4. A system as in claim 1, wherein the seal of the port prevents entry of fluid into the catheter until the seal is broken.

5. A system as in claim 1, wherein the blood vessel is an umbilical vein.

6. A system as in claim 1, wherein the seal is a septum.

7. A system as in claim 1, wherein the septum is made of a material that can be pierced by a cannula to provide access to an internal reservoir of the access port.

8. A method of accessing an umbilical vein, comprising:
cutting an umbilical cord of a patient to expose a blood vessel of the umbilical cord;
inserting a catheter into the blood vessel, wherein the catheter includes an access port located on a distal region of the catheter, wherein the access port includes a seal that provides access to a lumen of the umbilical vessel via a catheter lumen of the catheter;
maintaining the access port external to the patient;
treating the blood vessel via the catheter.

9. A method as in claim 1, wherein the blood vessel is an umbilical vein.

10. A method as in claim 1, further comprising attaching the access port to the catheter prior to inserting the catheter into the blood vessel.

11. A method as in claim 1, wherein the access port is pre-attached to the catheter prior to inserting the catheter into the blood vessel.

12. A method as in claim 11, wherein a user does not attach the access port to the catheter.

13. A method as in claim 11, wherein a user must break the seal of the access port to gain access to the blood vessel via the catheter.
